(19) United States
(12) Patent Application Publication
(10) Pub. No.: US 2024/0341888 A1
(43) Pub. Date: Oct. 17, 2024

(54) DIGITAL HANDING USING A FLEX OR BEND SENSOR

(71) Applicants: Daniel S. Menoher, Hummelstown, PA (US); Kevin Andrew Hufford, Durham, NC (US); Randall L. Luck, Cary, NC (US); Shawn M. Pierpont, Durham, NC (US)

(72) Inventors: Daniel S. Menoher, Hummelstown, PA (US); Kevin Andrew Hufford, Durham, NC (US); Randall L. Luck, Cary, NC (US); Shawn M. Pierpont, Durham, NC (US)

(21) Appl. No.: 18/582,642

(22) Filed: Feb. 20, 2024

Related U.S. Application Data
(60) Provisional application No. 63/486,004, filed on Feb. 20, 2023.

(51) Int. Cl. A61B 34/00; A61B 34/20

(52) U.S. Cl. CPC A61B 34/74; A61B 2034/2061

(57) ABSTRACT

A surgeon input tool is configured to generate input to a surgical robotic system to actuate jaw open-close of a surgical instrument. The tool comprises a grip and an elongate member extending from the grip such that when the grip is positioned against a palm of a human hand, the elongate member is positionable in contact with a finger of the hand, such as the index finger. The member includes a flexible bend sensor which generates signals in response to bending of the member by the user.

### REFERENCE TO RELATED APPLICATIONS

This application claims the benefit of U.S. Provisional Application No. 63/486,004, filed Feb. 20, 2023.

### TECHNICAL FIELD OF THE INVENTION

The present invention relates generally to the field of robot-assisted surgical devices and systems, and more particularly to devices and systems for providing user input to surgical robotic systems to cause corresponding movement of surgical instruments at a surgical site.

### BACKGROUND

There are various types of surgical robotic systems on the market or under development. Some surgical robotic systems use a plurality of robotic arms. Each arm carries a surgical instrument, or the camera used to capture images from within the body for display on a monitor. Typical configurations allow two or three instruments and the camera to be supported and manipulated by the system. Input to the system is generated based on input from a surgeon positioned at a master console, typically using input devices such as input handles. Motion and actuation of the surgical instruments and the camera is controlled based on the user input. The image captured by the camera is shown on a display at the surgeon console. The console may be located patient-side, within the sterile field, or outside of the sterile field.

The robotic arms/manipulators include a portion, typically at the terminal end of the arm, that is designed to support and operate a surgical device assembly. The surgical device assembly includes a surgical instrument having a shaft and a distal end effector on the shaft. The end effector is positionable within a patient. The end effector may be one of many different types that are used in surgery including, without limitation, end effectors having one or more of the following features: jaws that open and close, a section at the distal end of the shaft that bends or articulates in one or more degrees of freedom, a tip that rolls axially relative to the shaft, a shaft that rolls axially relative to the manipulator arm.

In many robotic surgical systems, particularly those using rigid shaft instruments, the surgical instruments are both robotically manipulated by the robotic manipulator arms disposed outside the patient's body, as well as electromechanically actuated within the patient's body. Robotic manipulation pivots the instrument shaft relative to the patient and may alter the insertion depth of the instrument and/or cause the instrument to roll about its longitudinal axis. Electromechanical actuation (or hydraulic/pneumatic actuation) may open and close jaws of the instrument, and/or actuate articulating or bending of the distal end of the instrument shaft, and/or roll the instrument's shaft or distal tip. Some systems may use only this latter form of instrument motion while holding the more proximal part of the instrument in a fixed position outside the body using a fixed support or inactive robotic manipulator.

For surgical instruments that are actuated to carry out jaw open-close, shaft articulating or bending, or other functions, there is typically a mechanical interface between the adapter and the robotic manipulator through which motion generated by the instrument actuators within the robotic manipulator is communicated to one or more mechanical inputs of the adapter to control any degrees of freedom of the instrument and, if applicable, its jaw open-close function. This motion may be communicated through a drape positioned between the sterile adapter and the non-sterile manipulator arm. In some commercially available robotic systems, the motion is communicated using rotary connections in which rotating disks on the manipulator transfer motion to rotating disks on an instrument adapter, while in others it is communicated using linearly-translating elements.

The instruments are exchangeable during the course of the procedure, allowing one instrument (with its corresponding adapter) to be removed from a manipulator and replaced with another. To minimize surgical procedure time and make efficient use of operating room personnel, it is essential to make the instrument exchange process as efficient as possible.

The number of degrees of freedom (DOFs) of motion for a robotically controlled instrument can vary between surgical systems and also between the different devices used for a particular system. Likewise, instruments with varying levels of complexity can be used interchangeably on a particular type of robotic system.

For example, a robotically controlled rigid-shafted instrument that moves similarly to a conventional laparoscopic instrument will be pivoted by the robotic arm relative to a fulcrum at the incision site (instrument pitch-yaw motion), axially rolled about the instrument's longitudinal axis, and translated along the longitudinal axis of the instrument (along the axis of insertion/withdrawal of the instrument relative to the incision). Other robotically controlled rigid-shafted instruments might be configured to move in a manner similar to that described in the previous paragraph, but to also have slightly more complexity, such as one or more degrees of articulation of the end effector about the instrument shaft (e.g. pitch and jaw of the instrument end effector relative to the shaft, which may be in addition to the pitch and/or yaw that results from movement of the rigid instrument shaft about a fulcrum at the incision site), giving the instrument 6DOFs. See, for example, the instruments described in co-pending and commonly owned application US 2020/0315722, Articulating Surgical Instrument, which is incorporated by reference. Such instruments might optionally also include the ability to axially roll the instrument's tip about the shaft.

There are other types of user instrument handle motion, besides laparoscopic motion, used in surgery. Another type of instrument handle motion used in surgery is referred to as “true cartesian motion,” which differs from laparoscopic motion in that there is no inversion of the motion, so the user input handle is raised to cause the surgical robotic system to raise the instrument tip, moved left to cause movement of the tip to the left, etc. Some surgical systems may allow surgical personnel to choose whether the system will operate in a laparoscopic type of mode or in a true cartesian motion mode. Others might make use of a surgeon console that is configured so it can be selectively used use with a laparoscopic surgical system and with a true cartesian surgical system.

Digital handles may be used to capture surgeon gestures to be translated into surgical intent for the robotic system. The present application describes a lean handle that is intended to be tethered to one's hand and mimic natural hand motions. The disclosed design utilizes a flex sensor to enable the user to provide surgeon intent through a minimalistic handle design that significantly reduces the handle's form factor. The flex sensor also translates the desired jaw closure gestures in a natural movement that significantly simplifies the user interface, reducing cognitive load. The handles shown herein are intended to be used as input devices to capture surgeon motion for a robotic surgical system.

The described handle may be used with a variety of handle tracking modalities, including optical, electromagnetic, inertial, or any combination of the above. It may also be used in the hand tracking system described in co-pending application No. PCT/US24/16576, entitled Surgeon Input System Using Event-Based Vision Sensors for a Surgical Robotic System.

The digital handle also contains haptic actuators that provide feedback to the user during use.

### BRIEF DESCRIPTION OF THE DRAWINGS

FIG. 1 is a perspective view of a trackable surgeon input tool using a flexible bend sensor.

FIGS. 2 and 3 show examples of bend sensors suitable for the described embodiments.

FIGS. 4A, 4B and 5 show a second embodiment of a trackable surgeon input tool using a flexible bend sensor.

FIGS. 6 and 7 show examples of 2D bend sensors.

### DETAILED DESCRIPTION

### Flexible Sensor Implementation

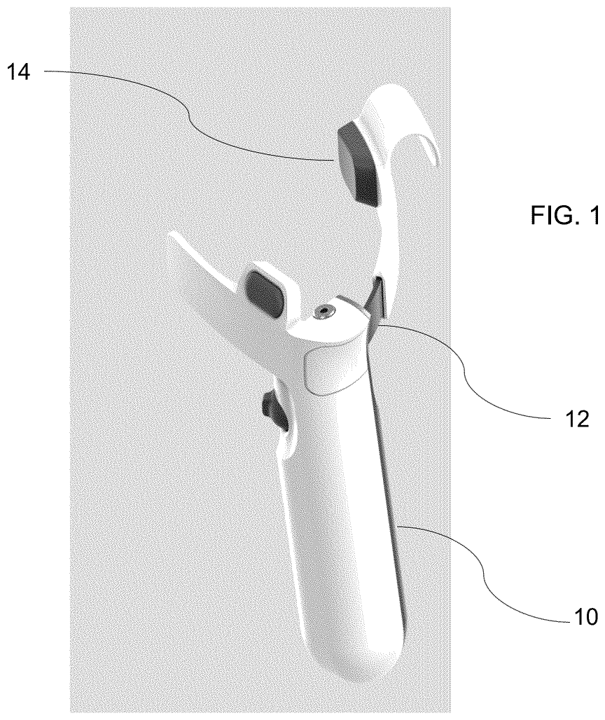

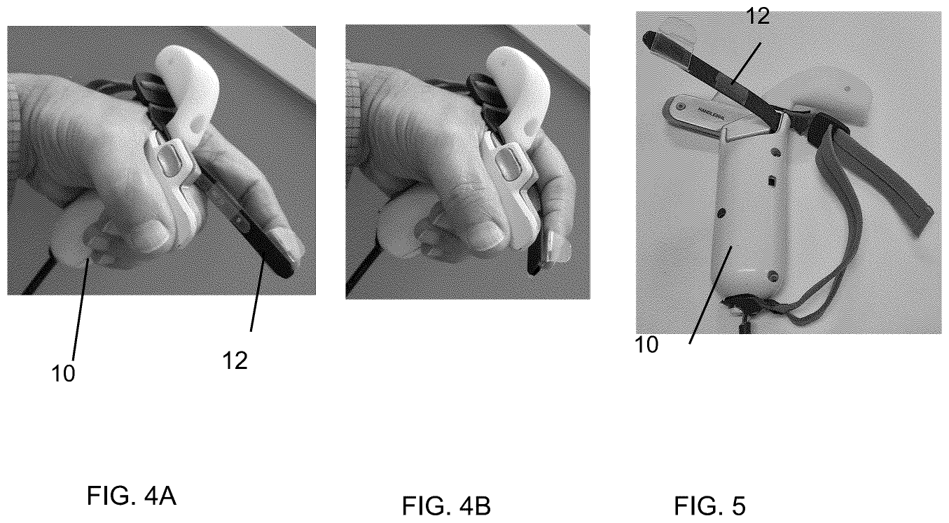

FIGS. 1 and 4A illustrate examples of low-profile digital handles having features moveable in a way that mimics natural hand motions. More specifically, the handles are provided with a handle or grip portion 10 positionable against a user's palm, and a flex element 12 positioned to be moved by a user's finger, such as the index finger as shown in FIG. 4. The configuration allows the user to move the flex element with the relevant finger to communicate the user's intent to have the robotic surgical system close the jaws of a surgical instrument that is being manipulated by the robotic system.

FIGS. 4A and 4B illustrate an exemplary handle constructed using a resistive flex sensor. In FIG. 4A, the resistive flex sensor is in a first position corresponding to an open jaw position. In FIG. 4B, the resistive flex sensor is in a second position corresponding to a closed jaw position. Here, movement of the user's index finger in a trigger-pull motion moves the flex sensor from the first position to the second position.

There are several implementations of the flex sensor that provide different responses. A single axis flex sensor can be used for jaw closure, while a multi-axis flex sensor can provide jaw closure plus additional translational motion to robotic control.

### 1-D Flex Sensor Configurations:

When moved in the manner described above, a one-dimensional resistive flex sensor provides single axis jaw closure measurement that can be used by the system to initiate translation of jaw closure of the instrument tip. The jaw closure translation algorithm allows the robotic laparoscopic instrument to proportionally track the opening and closing of the handle bending.

Resistive flex sensors operate via a change in resistance vs. the amount of flex. As the flex sensor is bent further, the overall resistance increases. In one configuration, one lead of the flex sensor is connected to ground, and the other lead of the flex sensor is connective to the + (positive) input of an operational amplifier. An external fixed resistor is placed between the same + input of the operational amplifier and the supply voltage (e.g. a +3.3V supply voltage). These two resistances form a variable voltage divider. The remainder of the operational amplifier is connected as a gain stage. The flex sensor varies between about 25 K ohms when it is straight and 50 K ohms when it is fully bent. The resistor to +3.3V is 22 K, thus via the voltage divider equation, the voltage at the +input can vary between about +1.75V and about +2.30V for a total span of about 0.55V. The operational amplifier circuit is configured with a gain of 4.7 and offset appropriately so that its output ranges from about +0.5V and about +3.0V. This is then sampled with an analog to digital converter in a microcontroller. This microcontroller data is then forwarded to the robotic control system where it is used to control the laparoscopic instrument jaws.

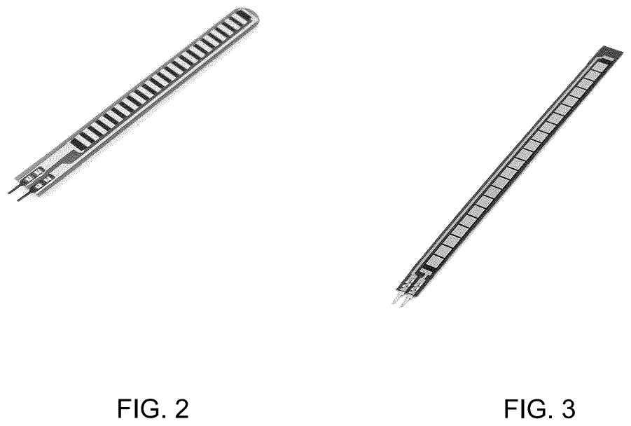

Example resistive flex sensors are shown in FIGS. 2 and 3 and can be seen at https://www.spectrasymbol.com/product/flex-sensors/

Another type of flex sensor is available from Bend Labs. This is a component that has a flexible silicone rubber membrane with a capacitance pattern embedded in it. As the rubber is bent the capacitance changes. The component includes integrated electronics circuitry that interprets this capacitance change such that it eventually outputs a digital signal in the I2C format that is proportional to the total amount of bending. The microcontroller in the handle then reads this I2C data to obtain the amount of bending. This microcontroller data is then forwarded to the robotic control system where it is used to control the laparoscopic instrument jaws.

Other suitable bend sensors can be found at https://www.nitto.com/us/en/nbt/products/index.html

### 2-D Flex Sensor Configurations:

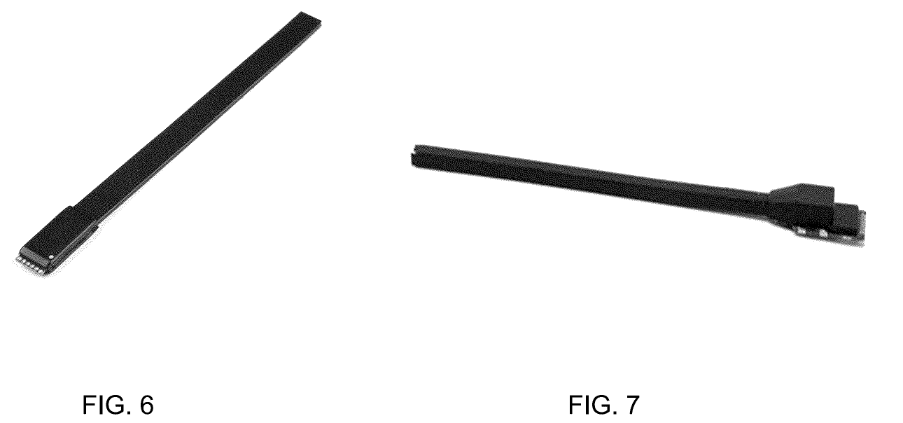

In some implementations, a flexible 2-D sensor is integrated into the surgeon input tool. 2D flex sensors are illustrated in FIGS. 6 and 7. As discussed, a multi-axis bend sensor can provide jaw closure plus additional translational motion to robotic control.

Thus, for example, with a 2-axis flexible sensor, side-to-side motion may be used to cue scrolling motions, rolling of the instrument along its axis, etc.

### Physical Constraints

In some implementations, the bend sensor is allowed to bend smoothly along its entire length. In others, mechanical features of the handle may be used to guide the bending, at least slightly localizing the bend location(s) for improved performance or better user immersion.

In general, in a preferred handle the flex or bend sensor is placed on the inside of a strip of spring steel or similar resilient material that allows it to return to its natural biased state once pressure against it is released by a user.

### Haptics On Distal Tip

A key advantage of the digital handle design is the ability to embed haptic actuators in the region of the finger tips. Haptics in the handle tips increases sensitivity to the user when touch related stimuli is desired.

### Haptics in Palm

Additionally Haptic actuators have been added in the palm region. Haptics in this region are used to pass information to the user such as arm collisions, approaching an edge of tracked space, and or abdominal wall haptics.

1. A surgeon input tool comprising:
a grip, and
an elongate member extending from the grip such that when the grip is positioned against a palm of a human hand, the elongate member is positionable in contact with a finger of the hand, wherein the member comprises a flexible bend sensor and is moveable relative to the grip between a first position and a second position.

2. The surgeon input tool of claim 1, wherein the flexible bend sensor is configured to sense bending in 1DOF.

3. The surgeon input tool of claim 1, wherein the bend sensor is configured to send bending in 2 DOFs.

4. The surgeon input tool of claim 1, further including at least one haptic element disposed on at least one of the grip and the elongate member.

5. The surgeon input tool of claim 1, further including a spring member resiliently biasing the flexible bend sensor in the first position.

6. A method of generating input for commanding jaw actuation of a robotically controlled surgical instrument, the method including:
positioning a surgeon input tool in contact with a hand of a surgeon,
using a finger of the hand, applying a force to a flexible bend sensor of the surgeon input tool in a first direction, causing said bend sensor to bend and generate a first signal, and
in response to the first signal, causing a jaw of the surgical instrument to move from an open position to a closed position.

7. The method of claim 6, wherein the method further includes:
bending the flexible bend sensor in a second direction different from the first direction, causing said bend sensor to generate a second signal, and
in response to the second signal, causing axial rolling of the surgical instrument relative to its longitudinal axis.

8. The method of claim 6, wherein the flexible bend sensor is moveable from a first position to a second position to generate the first signal, and wherein the method includes resiliently moving the flexible bend sensor from the second position to the first position upon at least partial release of the force.
